(19) United States
(12) Patent Application Publication
(10) Pub. No.: US 2026/0287507 A1
(43) Pub. Date: Sep. 24, 2026

(54) DETECTION DEVICE

(71) Applicant: Japan Display Inc., Tokyo (JP)

(72) Inventors: Kaoru ITO, Tokyo (JP); Norio MAMBA, Tokyo (JP); Daichi ABE, Tokyo (JP); Yoshihiro SEKIGUCHI, Tokyo (JP)

(21) Appl. No.: 19/570,913

(22) Filed: Mar. 18, 2026

(30) Foreign Application Priority Data
Mar. 24, 2025 (JP) 2025-048883

(51) Int. Cl. G01N 21/59; C12M 1/34

(52) U.S. Cl. CPC G01N 21/59; C12M 41/36

(57) ABSTRACT

According to an aspect, a detection device includes: a housing; a light source configured to emit light; a planar optical sensor where a plurality of optical sensors configured to detect the light from the light source are two-dimensionally arranged; and an object placement part on which an object to be detected is allowed to be placed such that the object to be detected is interposed between the light source and the planar optical sensor. The planar optical sensor, the object placement part, and the light source are arranged in the housing in the order as listed. A heater is provided in the housing.

### CROSS-REFERENCE TO RELATED APPLICATION

This application claims the benefit of priority from Japanese Patent Application No. 2025-048883 filed on Mar. 24, 2025, the entire contents of which are incorporated herein by reference.

### BACKGROUND

### 1. Technical Field

What is disclosed herein relates to a detection device.

### 2. Description of the Related Art

Detection devices are known that enable detection of states of culture environments for culturing biological tissues or microorganisms using an optical sensor (for example, Japanese Patent Application Laid-open Publication No. 2005-087005).

When detecting the states of culture environments using the optical sensor, a plurality of the optical sensors are two-dimensionally arranged and the culture environments are planarly detected. Since such a detection device does not have a temperature regulation function, the whole device needs to be accommodated in a thermostatic chamber or an incubator, and controlled in temperature. In this case, wiring needs to be coupled thereto from outside in order to transfer data and supply power. As a result, cost can increase.

For the foregoing reasons, there is a need for a detection device that can suppress an increase in cost.

### SUMMARY

According to an aspect, a detection device includes: a housing; a light source configured to emit light; a planar optical sensor where a plurality of optical sensors configured to detect the light from the light source are two-dimensionally arranged; and an object placement part on which an object to be detected is allowed to be placed such that the object to be detected is interposed between the light source and the planar optical sensor. The planar optical sensor, the object placement part, and the light source are arranged in the housing in the order as listed. A heater is provided in the housing.

### BRIEF DESCRIPTION OF THE DRAWINGS

FIG. 1 is a diagram illustrating a main configuration of a detection device;

FIG. 2 is a diagram illustrating a configuration example of a detection area and a wiring area;

FIG. 3 is a circuit diagram illustrating a circuit configuration of an optical sensor;

FIG. 4 illustrates schematic views illustrating configuration examples of a light source;

FIG. 5 is a schematic view illustrating a structure of various parts including main components of the detection device and an object to be detected that is placed on the detection device;

FIG. 6 is a schematic plan view illustrating a case where the object to be detected is viewed from a planar optical sensor side while being placed on a light-transmitting member;

FIG. 7 is a schematic plan view illustrating a case where the detection device illustrated in FIG. 5 is viewed from the planar optical sensor side;

FIG. 8 is a schematic view illustrating a structure of various parts including main components of a detection device according to a second embodiment of the present disclosure and the object to be detected that is placed on the detection device;

FIG. 9 is a schematic plan view illustrating a case where the detection device illustrated in FIG. 8 is viewed from the planar optical sensor side;

FIG. 10 is a schematic view illustrating a structure of various parts including main components of a detection device according to a third embodiment of the present disclosure and the object to be detected that is placed on the detection device;

FIG. 11 is a schematic plan view illustrating a light-emitting area of a light-emitting panel illustrated in FIG. 10; and

FIG. 12 is a schematic perspective view illustrating a heater, a dish, and the planar optical sensor according to a fourth embodiment of the present disclosure.

### DETAILED DESCRIPTION

The following describes embodiments of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

### First Embodiment

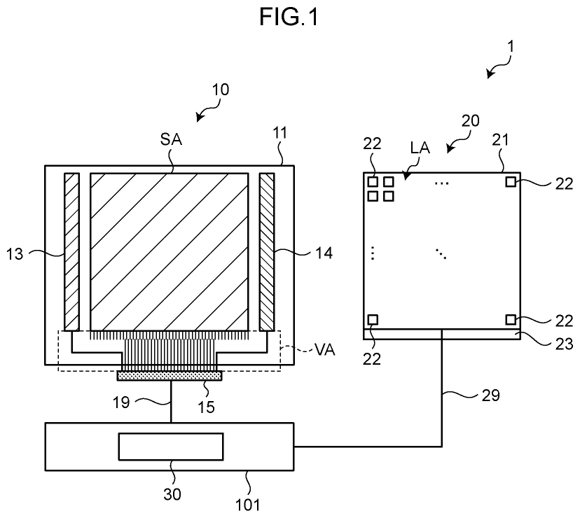

FIG. 1 is a diagram illustrating a main configuration of a detection device 1. The detection device 1 includes a planar optical sensor 10, a light source panel 20, and a control circuit 30. The planar optical sensor 10 and the light source panel 20 of the detection device 1 are coupled to a control board 101. The control board 101 is provided with a control circuit 102 and other components.

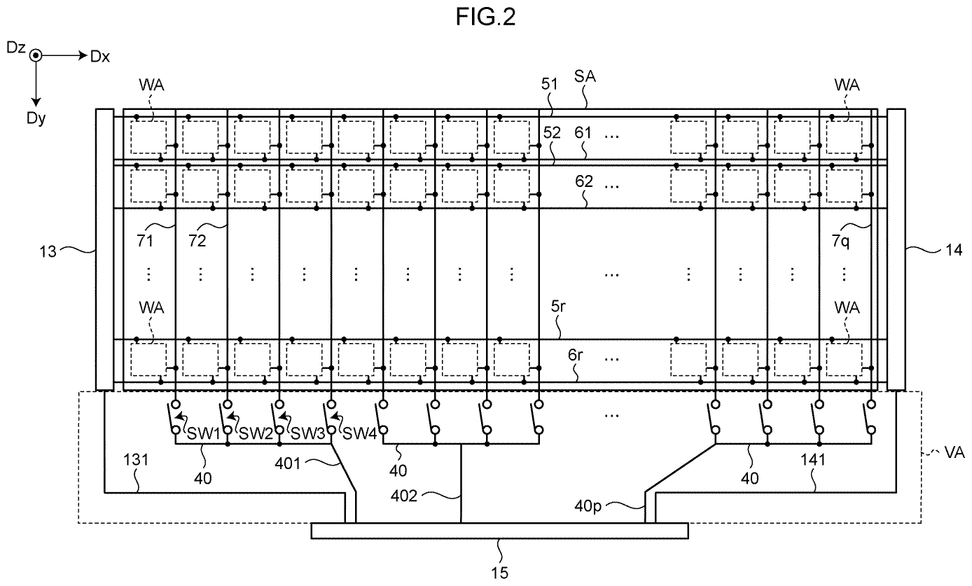

The planar optical sensor 10 is provided with a detection area SA (refer to FIG. 2) on a substrate 11. A reset circuit 13, a scan circuit 14, and a wiring area VA are provided on the substrate 11. Components on the detection area SA, the reset circuit 13, and the scan circuit 14 are coupled to a detection circuit 15 via the wiring area VA.

The light source panel 20 has a light-emitting area LA that emits light to the detection area SA. The light source panel 20 is provided with a light source 22 on a substrate 21. The light source 22 includes a light-emitting element, such as a light-emitting diode (LED) and is provided in the light-emitting area LA. In the example illustrated in FIG. 1, a plurality of the light sources 22 are arranged in a matrix having a row-column configuration on the substrate 21.

The light source panel 20 is provided with a light source drive circuit 23. Under the control of the control circuit 30, the light source drive circuit 23 controls turning on and off of each of the light sources 22 and the luminance thereof when being turned on. The light sources 22 may be provided to allow individual control of light emission or may be provided so as to emit light collectively.

The control circuit 30 performs various processes related to operations of the detection device 1. Specifically, the control circuit 30 is a circuit, such as a field-programmable gate array (FPGA) or an application-specific integrated circuit (ASIC) that can implement a plurality of functions. The control circuit 30 is coupled to the detection circuit 15 via wiring 19 and obtains an output from the detection circuit 15. The control circuit 30 is coupled to the light source drive circuit 23 via wiring 29, and performs processing related to the lighting of the light sources 22, such as determination of lighting patterns of the light sources 22.

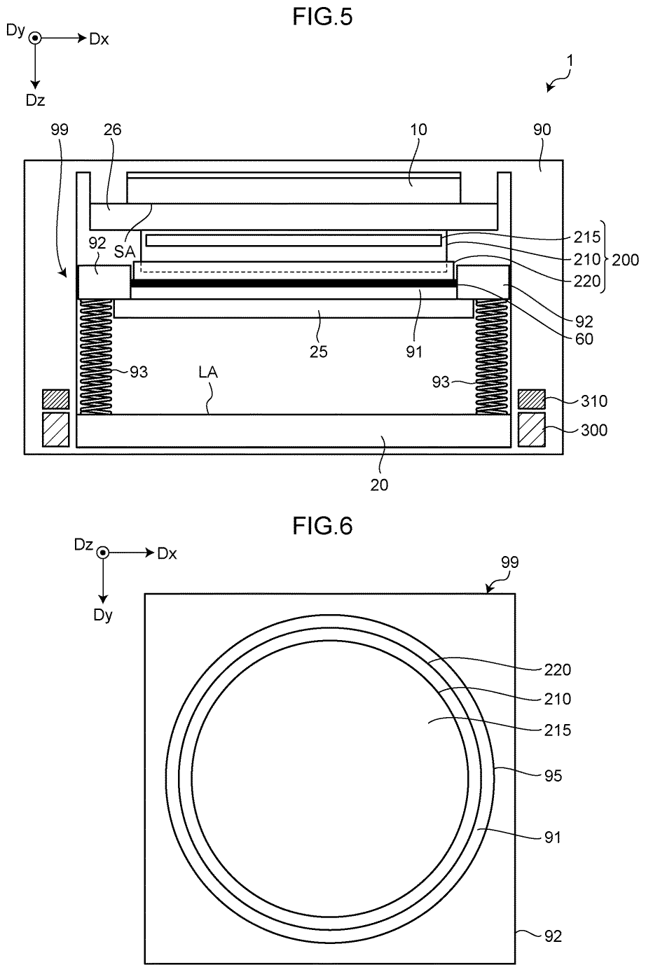

The control circuit 30 also performs processing related to detection of a colony in an object to be detected 200 (refer to FIG. 5) to be described later.

Although not illustrated in the drawings, the detection device 1 includes an analog-to-digital conversion circuit, a digital-to-analog conversion circuit, and other components. The analog-to-digital conversion circuit allows an output from an optical sensor WA (refer to FIG. 2) transmitted through the detection circuit 15 to be handled by arithmetic processing by the control circuit 30. The digital-to-analog conversion circuit allows digital signals generated by the arithmetic processing of the control circuit 30 to be used for controlling operations of the planar optical sensor 10 and the light source panel 20. These circuits may be included, for example, in part or in whole in the control circuit 30, may be functions performed by circuits mounted on flexible printed circuits (FPCs) provided as the wiring 19 and the wiring 29, or may be implemented in other ways in the detection device 1.

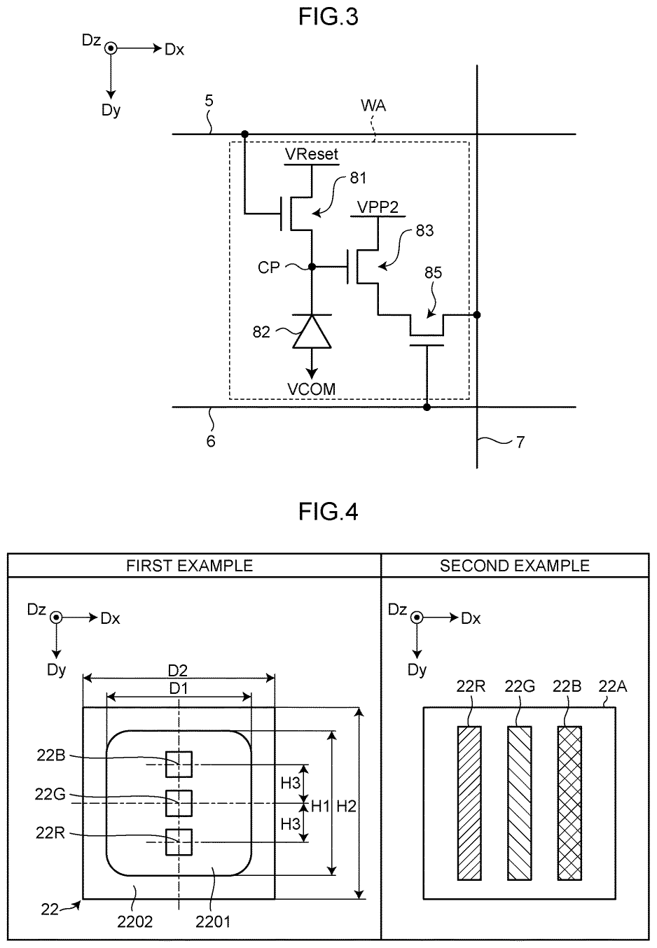

FIG. 2 is a diagram illustrating a configuration example of the detection area and the wiring area. A plurality of the optical sensors WA (FIG. 3) are provided in the detection area SA. In the first embodiment, as illustrated in FIG. 2, the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset signal transmission lines 51, 52, . . . , 5r. Hereinafter, the term “reset signal transmission line 5” refers to any one of the reset signal transmission lines 51, 52, . . . , 5r. The reset signal transmission line 5 is wiring along the first direction Dx. In the example illustrated in FIG. 2, r reset signal transmission lines 5 are arranged in the second direction Dy. r is a natural number equal to or larger than 2. The r reset signal transmission lines 5 are each coupled, at one end in the first direction Dx, to the reset circuit 13.

The scan circuit 14 is coupled to scan lines 61, 62, . . . , 6r. Hereinafter, the term “scan line 6” refers to any one of the scan lines 61, 62, . . . , 6r. The scan line 6 is wiring along the first direction Dx. In the example illustrated in FIG. 2, r scan lines 6 are arranged in the second direction Dy. The r scan lines 6 are each coupled, at the other end in the first direction Dx, to the scan circuit 14.

As illustrated in FIG. 2, the reset signal transmission lines 5 and the scan lines 6 are alternately arranged in the second direction Dy in the detection area SA. The reset circuit 13 and the scan circuit 14 illustrated in FIGS. 1 and 2 are arranged at locations facing each other with the detection area SA interposed therebetween, but the layout of the reset circuit 13 and the scan circuit 14 is not limited to this layout and can be changed as appropriate.

Signal lines 71, 72, . . . , 7q are also provided in the detection area SA. Hereinafter, the term “signal line 7” refers to any one of the signal lines 71, 72, . . . , 7q. The signal line 7 is wiring along the second direction Dy.

In the example illustrated in FIG. 2, q signal lines 7 are arranged in the first direction Dx. q is a natural number equal to or larger than 2. The q signal lines 7 are each coupled, at one end in the second direction Dy, to one of a plurality of switches (for example, switch SW1, SW2, SW3, or SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in one multiplexer 40 is on (conducting state), the other switches are off (non-conducting state). The number of the multiplexers 40 depends on the number (q) of the signal lines 7. When the number of the switches is p, q/p is sufficient as the number of the multiplexers 40. When multiple multiplexers 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the scan circuit 14 is coupled to the detection circuit 15 via wiring 141.

In detecting light using a PD 82 (refer to FIG. 3) provided in the optical sensor WA, the detection circuit 15 controls operation timing of the reset circuit 13 and the scan circuit 14. The detection circuit 15 receives the outputs from the optical sensors WA. The detection circuit 15 converts the signals received from the optical sensors WA into data that can be interpreted by the control circuit 30 and outputs the data to the control circuit 30. The detection circuit 15 of the first embodiment is a microcontroller unit (MCU).

FIG. 3 is a circuit diagram illustrating a circuit configuration of the optical sensor WA. The first direction Dx and the second direction Dy in FIG. 3 merely correspond to the directions of the reset signal transmission lines 5, the scan lines 6, and the signal lines 7, and do not exactly indicate the relative positional relation of the circuit configuration in the optical sensor WA.

As illustrated in FIG. 3, a switching element 81, the PD 82, a transistor element 83, and a switching element 85 are provided in the optical sensor WA. The PD 82 is a photodiode (PD). The switching elements 81 and 85 and the transistor element are metal-oxide semiconductor field-effect transistors (MOSFETs).

The gate of the switching element 81 is coupled to the reset signal transmission line 5. One of the source and the drain of the switching element 81 is supplied with a reset potential VReset. The other of the source and the drain of the switching element 81 is coupled to the cathode of the PD 82 and the gate of the transistor element 83. Hereinafter, the term “coupling part CP” refers to a point where the other of the source and the drain of the switching element 81 is coupled to the cathode of the PD 82 and the gate of the transistor element 83. A reference potential VCOM is supplied from the anode side of the PD 82. The potential difference between the reset potential VReset and the reference potential VCOM is set in advance, but the reset potential VReset and the reference potential VCOM may be variable. The reset potential VReset is higher than the reference potential VCOM.

The drain of the transistor element 83 serving as a source follower is supplied with an output source potential VPP2. The source of the transistor element 83 is coupled to one of the source and the drain of the switching element 85. The other of the source and the drain of the switching element 85 is coupled to the signal line 7. The gate of the switching element 85 is coupled to the scan line 6.

The reset potential VReset, the reference potential VCOM, and the output source potential VPP2 are supplied by the detection circuit 15 to the optical sensor WA based on, for example, electric power supplied via a power supply circuit (not illustrated) coupled to the detection circuit 15. The way of outputting these potentials is not limited to this way and can be changed as appropriate.

The output source potential VPP2 is set in advance. The potential on the source side of the transistor element 83 is a potential lower than the output potential of the PD 82 by a voltage (Vth) between the gate and the source of the transistor element 83. In this case, the potential on the source side of the transistor element 83 corresponds to the reset potential VReset and the reference potential VCOM. The potential of the output of the PD 82 corresponds to photovoltaic power generated by the PD 82 in response to the light detected by the PD 82 during an exposure period.

When the gate of the switching element 85 is turned on by the gate signal supplied from the scan circuit 14 via the scan line 6, the source and the drain of the switching element 85 are brought into a conducting state therebetween. This operation transmits, to the signal line 7 via the switching element 85, a signal (potential) transmitted via the transistor element 83 to the switching element 85. Thus, the output from the optical sensor WA is generated. Hereinafter, the term “gate signal” refers to the signal (potential) supplied from the scan circuit 14 via the scan line 6. The scan circuit 14 is a circuit that outputs the gate signal. As described with reference to FIGS. 2 and 3, the optical sensors WA coupled to the scan lines 6 and the signal lines 7 are arranged in a matrix having a row-column configuration in the detection area SA of the planar optical sensor 10. The scan line 6 is provided along the first direction Dx and is configured to transmit the gate signal that causes the optical sensors WA to generate the outputs. The signal line 7 is configured to transmit the outputs of the optical sensors WA along the second direction Dy.

The output of one PD 82 provided in one optical sensor WA corresponds to the intensity of the light detected by the PD 82 during the exposure period set in advance. The output of the PD 82 is reset in response to a signal supplied by the reset circuit 13 via the reset signal transmission line 5. When the signal turns on the gate of the switching element 81, the source and the drain of the switching element 81 are brought into a conducting state therebetween. This operation resets the potential of the coupling part CP to the reset potential VReset.

FIG. 4 illustrates schematic views illustrating configuration examples of the light source. As illustrated in FIG. 4, the light source 22 includes a first light source 22R, a second light source 22G, and a third light source 22B. The first light source 22R, the second light source 22G, and the third light source 22B emit light in different colors from one another. In the first embodiment, the first light source 22R emits red (R) light. The second light source 22G emits green (G) light. The third light source 22B emits blue (B) light.

As illustrated as “First Example” in FIG. 4, the light source 22 has, for example, a light-emitting area 2201 and a frame area 2202. In the light-emitting area 2201, the first light source 22R, the second light source 22G, and the third light source 22B that are square in shape in plan view are arranged along the second direction Dy. The frame area 2202 is a frame-like area surrounding the light-emitting area 2201. A width D1 in the first direction Dx of the light-emitting area 2201 is smaller than a width D2 in the first direction Dx of the frame area 2202. A height H1 in the second direction Dy of the light-emitting area 2201 is smaller than a height H2 in the second direction Dy of the frame area 2202. The distance between the first light source 22R and the second light source 22G is a distance H3. The distance between the second light source 22G and the third light source 22B is also the distance H3. The distance H3 is less than half the height H1. In “First Example” in FIG. 4, the width D1 is equal to the height H1, and the width D2 is equal to the height H2, but at least one of the widths may differ from a corresponding one of the heights. The light source 22 may be replaced with a light source of another form, specifically, such as a light source 22A illustrated in “Second Example” in FIG. 4. In the light source 22A, the longitudinal directions of the first light source 22R, the second light source 22G, and the third light source 22B are along the second direction Dy, and the first light source 22R, the second light source 22G, and the third light source 22B are arranged in this order from one side toward the other side in the first direction Dx. First Example and Second Example in FIG. 4 are exemplary forms of the light source according to the present disclosure, which is not limited to these examples. The shapes of the first light source 22R, the second light source 22G, and the third light source 22B in plan view and the positional relation between the first light source 22R, the second light source 22G, and the third light source 22B can be changed as appropriate. The term “plan view” refers to a front view of a plane along the first direction Dx and the second direction Dy (Dx-Dy plane).

FIG. 5 is a schematic view illustrating a structure of various parts including main components of the detection device and the object to be detected that is placed on the detection device. The object to be detected 200 is a culture medium 215 (e.g., agar) accommodated in a dish 210 of a container. The container further includes a lid 220. The dish 210 is specifically a Petri dish. The lid 220 is a cover of the dish 210. As illustrated in FIG. 6 to be explained later, the inner diameter of an annular sidewall of the lid 220 is equal to or more than the outer diameter of the annular sidewall of the dish 210. That is, the lid 220 has a cylindrical inner circumferential wall that covers a cylindrical outer circumferential wall of the dish 210 from outside. The culture medium 215 is a culture medium capable of culturing the colony. Hereinafter, the term simply called “colony” refers to a colony of culture targets that have been cultured on the culture medium 215 of the object to be detected 200. The culture targets are, for example, biological tissues or microorganisms, which are assumed to be cultured on the culture medium 215. The culture medium 215 has a light-transmitting property with the degree of light transmission varying depending on the presence or absence of the colony and the thickness of the colony. The object to be detected 200 is placed on a light-transmitting member 91. The light-transmitting member 91 is a plate-like member made of colorless glass or a light-transmitting colorless synthetic resin.

FIG. 6 is a schematic plan view illustrating a case where the object to be detected is viewed from the planar optical sensor side while being placed on the light-transmitting member. As illustrated in FIG. 6, the light-transmitting member 91 is a circular member having a diameter that can accommodate therein the object to be detected 200 in plan view. The light-transmitting member 91 has a light-transmitting area that can accommodate therein the object to be detected 200 between the planar optical sensor 10 and the light source panel 20. The light-transmitting member 91 is in contact with a light-blocking member 92 at the outer peripheral edge. The light-blocking member 92 is a plate-like member into which the light-transmitting member 91 is fitted. The light-blocking member 92 has a light-blocking property.

An edge 95 illustrated in FIG. 6 is the outer peripheral edge of the light-transmitting member 91 and is the inner peripheral edge of the light-blocking member 92 into which the light-transmitting member 91 is fitted. The edge 95 is circular in plan view. A light-transmitting area may be formed inside the edge 95 by hollowing out the inside of the light-blocking member 92 in a circular shape so as to form an inner peripheral edge corresponding to the edge 95 and by placing a light-transmitting member serving as the light-transmitting member 91 over the light-blocking member 92. In this case, the light-transmitting member 91 need not have a circular disc shape.

In the first embodiment, a diffusion plate 25 is provided on the light source panel 20 side of the light-transmitting member 91. The diffusion plate 25 is an optical member that diffuses light. The diffusion plate 25 is arranged to be interposed between the light-transmitting member 91 and the light-emitting area LA of the light source panel 20. When the diffusion plate 25 receives the light emitted from the light-emitting area LA from the light source panel 20 side, the diffusion plate 25 further diffuses the traveling direction of the light as the light is transmitted toward the light-transmitting member 91. This diffusion can uniform, in plan view, the light from the light-emitting area LA formed by a set of the light sources 22 that are two-dimensionally arranged.

As illustrated in FIG. 5, in the first embodiment, an elastic member 93 is provided between the light-blocking member 92 and the light source panel 20. The elastic member 93 has elasticity to urge the light-blocking member 92 toward the planar optical sensor 10. Specifically, the elastic member 93 is a cylindrical compression coil spring, as illustrated, for example, in FIG. 5. The object to be detected 200 placed on the light-transmitting member 91 is pressed against a member 26 provided between the planar optical sensor 10 and the light-transmitting member 91 by an urging force applied to the light-blocking member 92 by the elastic member 93. In the first embodiment, an object placement part 99 is configured with the light-transmitting member 91, the light-blocking member 92, and the elastic member 93. In other words, the object placement part 99 includes the light-transmitting member 91 that is a light-transmitting member on which the object to be detected is placed, and the light-blocking member 92 that is a light-blocking member supporting the light-transmitting member from the outer periphery, and a boundary between the light-transmitting member 91 and the light-blocking member 92 has a circular shape like the edge 95 illustrated in FIG. 6. The diameter of the circle of the edge 95, that is, the boundary between the light-transmitting member 91 and the light-blocking member 92, is larger than the diameter of a circle of the edge of the dish 210.

The member 26 serves as an optical member that limits the light that is emitted from the light-emitting area LA of the light source panel 20 and reaches the planar optical sensor 10. Specifically, the member 26 includes any of a plate-shaped louver, cylindrical openings, and microlenses. In the plate-shaped louver, a plurality of plate-like structures with plate surfaces extending along the third direction Dz are arranged in parallel. The structures are preferably made of a material having a strong light-absorbing property. The member 26 is provided along a plane (Dx-Dy plane) orthogonal to the third direction Dz. The cylindrical openings penetrate the member 26 in the third direction Dz with respect to the base of the member 26. The base is preferably made of a material having a strong light-absorbing property. The microlenses are small lenses having an optical axis along the third direction Dz. The base of the member 26 that supports the microlenses is preferably made of a material having a strong light-absorbing property. Regardless of what shape the member 26 has, the member 26 as the optical member is provided in order to limit the traveling direction of the light emitted from the light sources 22 and reaching the planar optical sensor 10 to the third direction Dz or a direction having a shallower inclination angle with respect to the third direction Dz.

A housing 90 maintains a configuration in which the light-emitting area LA of the light source panel 20 and the detection area SA of the planar optical sensor 10 face in the third direction Dz. The housing 90 is a heat-insulating and light-blocking housing provided so as to accommodate therein in advance the light source panel 20, the elastic member 93, the diffusion plate 25, the light-transmitting member 91, the light-blocking member 92, the member 26, and the planar optical sensor 10. The planar optical sensor 10, the member 26, and the light sources 22 are arranged in this order in the housing 90. Placing the object to be detected 200 between the member 26 and the light-transmitting member 91 establishes the positional relation among the components illustrated in FIG. 5. In the first embodiment, the object to be detected 200 is placed on the light-transmitting member 91 so that the lid 220 side of the object to be detected 200 contacts the light-transmitting member 91. That is, the object to be detected 200 is placed between the planar optical sensor 10 and the light source panel 20 such that the lid 220 is arranged relatively below and the dish 210 is arranged relatively above.

As described above with reference to FIG. 5, the detection device 1 of the first embodiment has a structure that allows the object to be detected 200 to be placed so as to be interposed between the planar optical sensor 10 and the light source panel 20. In the placed object to be detected 200, the bottom surface of the dish 210 with the culture medium 215 therein extends along the detection area SA of the planar optical sensor 10 and the light-emitting area LA of the light source panel 20.

The light emitted from the light-emitting area LA of the light source panel 20 is diffused by the diffusion plate 25, passes through the light-transmitting member 91, the object to be detected 200, and the member 26, and reaches the detection area SA of the planar optical sensor 10. Thus, the planar optical sensor 10 can be said to be configured to output data reflecting the intensity of light that has been emitted from the light sources 22 and reached the optical sensors WA through the object to be detected 200. The data herein is data based on a set of the outputs from the optical sensors WA and can be regarded as data of an image. The image herein is obtained by regarding an output of one optical sensor WA as one pixel and arranging a plurality of the pixels so as to correspond to the arrangement of the optical sensors WA in the detection area SA. Hereinafter, the term simply called “image” refers to the set of the outputs of the optical sensors WA, unless otherwise noted. The term simply called “pixel” refers to the output of the optical sensor WA, unless otherwise noted. In practice, a process such as an analog-to-digital conversion is performed to regard the output of the optical sensor WA as the pixel. This process is performed by the detection circuit 15 in the first embodiment as described above, but may be performed by the control circuit 30.

The intensity of the light reaching the detection area SA is affected by the degree of light transmission of the culture medium 215. The uniformity of the light reaching the detection area SA is affected by the degree of diffusion of the light between the planar optical sensor 10 and the light source panel 20.

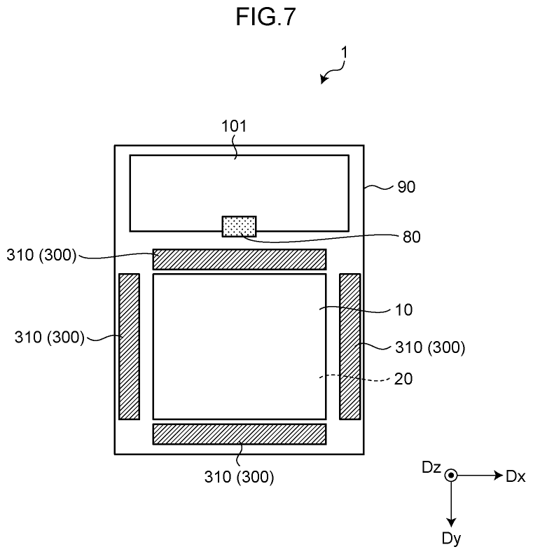

FIG. 7 is a schematic plan view illustrating a case where the detection device illustrated in FIG. 5 is viewed from the planar optical sensor side. As illustrated in FIGS. 5 and 7, heaters 300 are provided in the housing 90. When a current flows in each of the heaters 300, the heater 300 raises the surrounding temperature. The temperature of the object to be detected 200 changes depending on the temperature of the heater 300. For example, a resistance heating heater can be used as the heater 300. As illustrated in FIG. 7, the heaters 300 are arranged around at least one of the planar optical sensor 10, the object placement part 99, and the light source 22 in the housing 90 in plan view. As illustrated in FIGS. 5 and 7, the heaters 300 are arranged around the substrate 21.

As illustrated in FIG. 7, the control board 101 is provided with a temperature sensor 80. The temperature sensor 80 measures internal temperature of the housing 90 at intervals of one minute. The temperature sensor 80 controls the temperature of the heaters 300 using, for example, proportional-integral-derivative (PID) control or on-off control to set the internal temperature of the housing 90 to temperature optimal for culturing bacteria (for example, approximately 37°).

The heaters 300 may each be such that a material having high thermal conductivity is arranged between the dish 210 and an object to be heated of at least one of the planar optical sensor 10, the object placement part 99, and the light source 22.

As a result, an environment (temperature, humidity, and other factors) suitable for culturing the culture target on the object to be detected 200 can be maintained in the housing 90. Therefore, the heaters 300 can be used as a substitute for an incubator. Therefore, an increase in cost of the detection device 1 can be suppressed.

The detection device 1 further includes light-blocking members 310. As illustrated in FIGS. 5 and 7, the light-blocking members 310 are arranged adjacent to the heaters 300 in the housing 90.

This configuration can inhibit the light emitted from the heaters 300 from scattering and entering the planar optical sensor 10.

As illustrated in FIG. 5, the detection device 1 further includes a light directivity control element 60. The light directivity control element 60 is arranged between the object placement part 99 and the planar optical sensor 10. Specifically, the light directivity control element 60 is provided on the light-transmitting member 91. The light directivity control element 60 is an optical element that transmits, toward the PD 82, components of the light emitted from the light source panel 20, wherein the components is components traveling in a direction orthogonal to the planar optical sensor 10. The light directivity control element 60 is also called collimating apertures or a collimator. Alternatively, the light directivity control element 60 may be configured with a louver or microlenses instead of the collimator.

### Second Embodiment

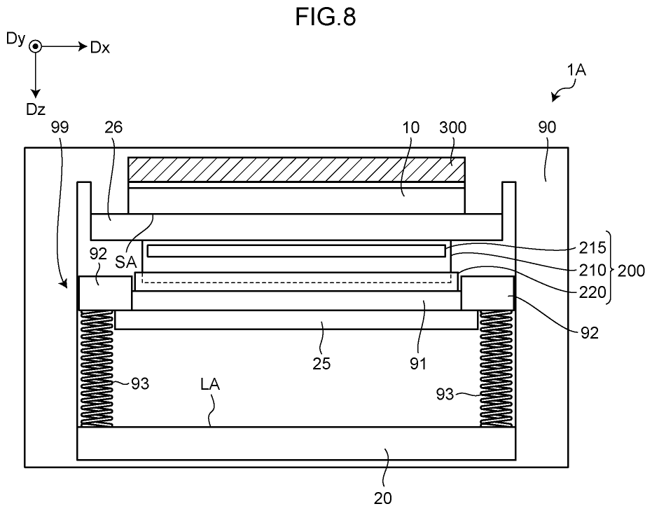

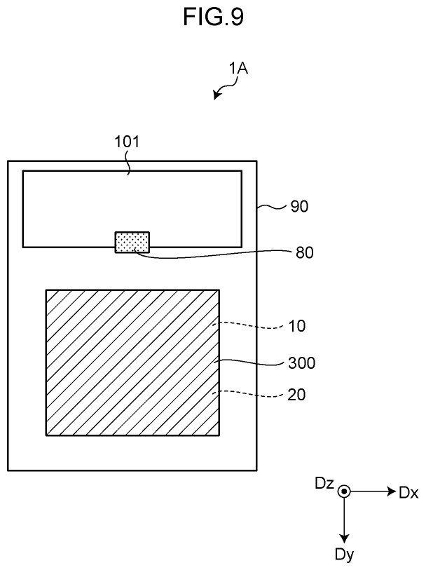

FIG. 8 is a schematic view illustrating a structure of various parts including main components of a detection device according to a second embodiment of the present disclosure and the object to be detected that is placed on the detection device. FIG. 9 is a schematic plan view illustrating a case where the detection device illustrated in FIG. 8 is viewed from the planar optical sensor side. In the following description, the same components as those described in the first embodiment described above are denoted by the same reference numerals, and the description thereof will not be repeated.

As illustrated in FIGS. 8 and 9, in a detection device 1A of the second embodiment, the heater 300 is arranged on the upper surface of the planar optical sensor 10 in plan view.

This configuration allows the environment (temperature, humidity, and other factors) in the housing 90 to be maintained to be suitable for culturing the culture target on the object to be detected 200. Therefore, the heater 300 can be used as a substitute for the incubator. Therefore, an increase in cost of the detection device 1A can be suppressed.

### Third Embodiment

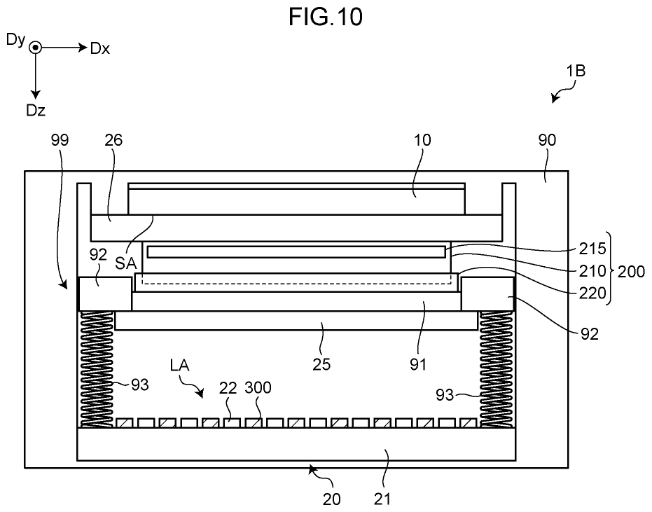

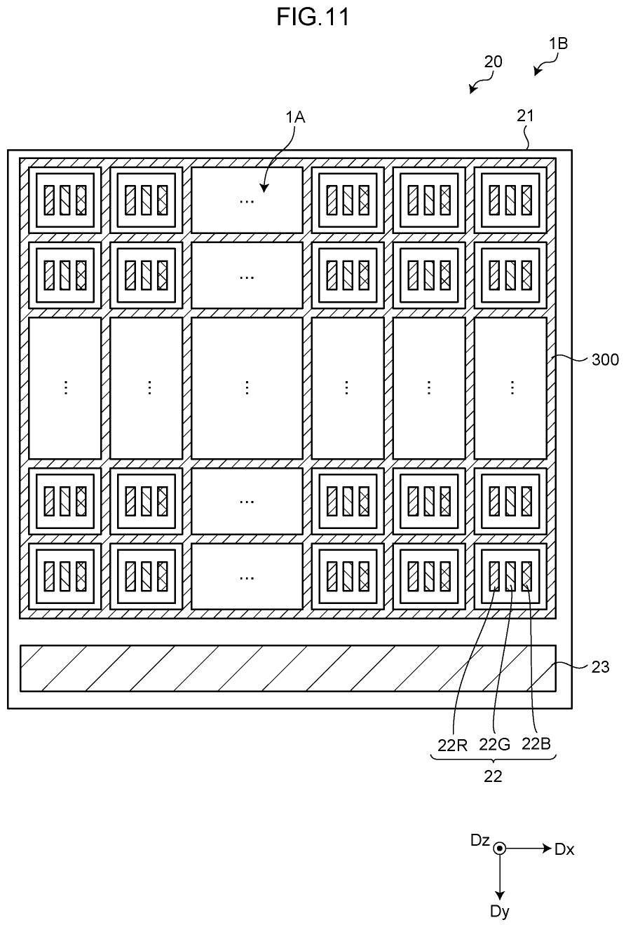

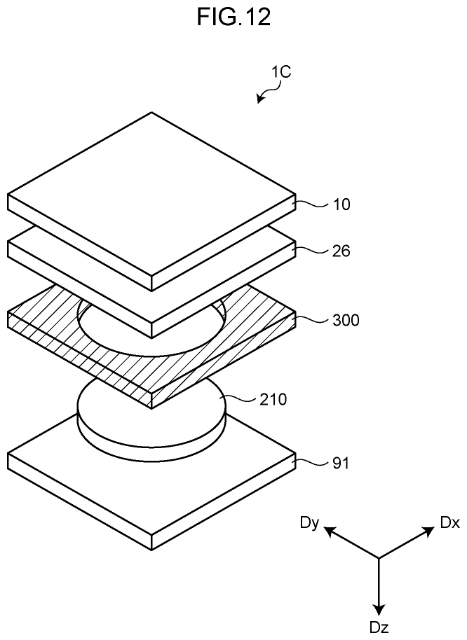

FIG. 10 is a schematic view illustrating a structure of various parts including main components of a detection device according to a third embodiment of the present disclosure and the object to be detected that is placed on the detection device. FIG. 11 is a schematic plan view illustrating a light-emitting area of a light-emitting panel illustrated in FIG. 10. In the following description, the same components as those described in the first embodiment described above are denoted by the same reference numerals, and the description thereof will not be repeated.

As illustrated in FIGS. 10 and 11, in a detection device 1B of the third embodiment, the heater 300 is arranged between the multiple light sources 22.

This configuration allows the environment (temperature, humidity, and other factors) in the housing 90 to be maintained to be suitable for culturing the culture target on the object to be detected 200. Therefore, the heater 300 can be used as a substitute for the incubator. Therefore, an increase in cost of the detection device 1B can be suppressed.

### Fourth Embodiment

FIG. 12 is a schematic perspective view illustrating the heater, the dish, and the planar optical sensor according to a fourth embodiment of the present disclosure. In the following description, the same components as those described in the first embodiment described above are denoted by the same reference numerals, and the description thereof will not be repeated.

As illustrated in FIG. 12, in a detection device 1C of the fourth embodiment, the heater 300 is provided between the member 26 and the light-transmitting member 91 and is arranged so as to surround the side surface of the dish 210.

This configuration allows the environment (temperature, humidity, and other factors) in the housing 90 to be maintained to be suitable for culturing the culture target on the object to be detected 200. Therefore, the heater 300 can be used as a substitute for the incubator. Therefore, an increase in cost of the detection device 1C can be suppressed.

Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a housing;
a light source configured to emit light;
a planar optical sensor where a plurality of optical sensors configured to detect the light from the light source are two-dimensionally arranged; and
an object placement part on which an object to be detected is allowed to be placed such that the object to be detected is interposed between the light source and the planar optical sensor, wherein
the planar optical sensor, the object placement part, and the light source are arranged in the housing in the order as listed, and
a heater is provided in the housing.

2. The detection device according to claim 1, wherein the heater is arranged around at least one of the planar optical sensor, the object placement part, and the light source in the housing in plan view.

3. The detection device according to claim 2, wherein
the light source comprises a plurality of light-emitting elements,
the light-emitting elements are arranged on a substrate, and
the heater is arranged around the substrate.

4. The detection device according to claim 2, wherein
the object to be detected is a culture medium accommodated in a dish of a container, and
the heater is arranged so as to surround a side surface of the dish.

5. The detection device according to claim 1, wherein
the light source comprises a plurality of light-emitting elements,
the light-emitting elements are arranged on a substrate, and
the heater is arranged between the light-emitting elements.

6. The detection device according to claim 1, wherein the heater is arranged on an upper surface of the planar optical sensor.

7. The detection device according to claim 1, further comprising a light-blocking member, wherein
the light-blocking member is arranged adjacent to the heater.

8. The detection device according to claim 1, further comprising a light directivity control element, wherein
the light directivity control element is arranged between the object placement part and the planar optical sensor.
